(19) United States
(12) Patent Application Publication
(10) Pub. No.: US 2025/0281131 A1
(43) Pub. Date: Sep. 11, 2025

(54) DATA PROCESSING APPARATUS, METHOD, AND PROGRAM

(71) Applicant: FUJIFILM Corporation, Tokyo (JP)

(72) Inventor: Jun YAMASHITA, Tokyo (JP)

(73) Assignee: FUJIFILM Corporation, Tokyo (JP)

(21) Appl. No.: 19/074,426

(22) Filed: Mar. 9, 2025

(30) Foreign Application Priority Data
Mar. 11, 2024 (JP) 2024-037205

(51) Int. Cl. A61B 6/42; G06T 5/40; G06V 10/25; G16H 30/20

(52) U.S. Cl. CPC A61B 6/4241; G06T 5/40; G06V 10/25; G16H 30/20

(57) ABSTRACT

There are provided data processing apparatus, method, and program which can improve accuracy of image selection. A data processing apparatus that processes data of a plurality of slices acquired by a spectral CT device includes a processor, in which the processor outputs a first image generated on the basis of the data to a display destination, receives setting of a region that is an analysis target, on the first image output to the display destination, analyzes a second image of the plurality of slices generated on the basis of the data to generate statistical information indicating a distribution of a material in the region for each slice, and outputs the statistical information to the display destination.

### CROSS-REFERENCE TO RELATED APPLICATION

The present application claims priority under 35 U.S.C § 119(a) to Japanese Patent Application No. 2024-037205 filed on Mar. 11, 2024, which is hereby expressly incorporated by reference, in its entirety, into the present application.

### BACKGROUND OF THE INVENTION

### 1. Field of the Invention

The present invention relates to data processing apparatus, method, and program, and particularly to data processing apparatus, method, and program for processing data obtained by a spectral computed tomography (CT) apparatus.

### 2. Description of the Related Art

As an X-ray CT device using energy information of the X-rays, a spectral CT device (multi-energy CT device) is known (for example, refer to JP2022-065390A, JP2018-139754A, JP2016-55164A, JP2015-144808A, and the like). From the data obtained by the spectral CT device, images having various kinds of information, such as a virtual monochromatic X-ray image, an effective atomic number image, and a material discrimination image, can be reconstructed.

### SUMMARY OF THE INVENTION

By the way, in an examination using the X-ray CT device, tens to thousands of images are captured in one examination. Therefore, there is a problem that it is difficult to check which slice has a symptom-specific material.

An embodiment according to the disclosed technology provides data processing apparatus, method, and program that can improve accuracy of image selection.

(1) A data processing apparatus that processes data of a plurality of slices acquired by a spectral CT device includes a processor, in which the processor outputs a first image generated on the basis of the data to a display destination, receives setting of a region that is an analysis target, on the first image output to the display destination, analyzes a second image of the plurality of slices generated on the basis of the data to generate statistical information indicating a distribution of a material in the region for each slice, and outputs the statistical information to the display destination.

(2) The data processing apparatus according to (1), in which the second image is a material discrimination image, and the processor analyzes the material discrimination image of the plurality of slices generated on the basis of the data to generate statistical information indicating a distribution of a density value of the material in the region for each slice.

(3) The data processing apparatus according to (2), in which the processor generates a histogram indicating an area of each density value in the region for each slice, as the statistical information.

(4) The data processing apparatus according to (2) or (3), in which the processor receives setting of a range of the density value to be displayed in the histogram, and generates the histogram within the set range of the density value.

(5) The data processing apparatus according to any one of (2) to (4), in which the first image is a material discrimination image, and the processor outputs the material discrimination image generated on the basis of the data to the display destination, and receives setting of the region on the material discrimination image output to the display destination.

(6) The data processing apparatus according to (1), in which the second image is an effective atomic number image, and the processor analyzes the effective atomic number image of the plurality of slices generated on the basis of the data to generate statistical information indicating a distribution of an effective atomic number in the region for each slice.

(7) The data processing apparatus according to (6), in which the processor generates a histogram indicating a count number of each effective atomic number in the region for each slice, as the statistical information.

(8) The data processing apparatus according to (6) or (7), in which the processor receives setting of a threshold value, and generates the histogram in which a portion corresponding to the count number equal to or greater than the threshold value is displayed in an emphasized manner.

(9) The data processing apparatus according to any one of (6) to (8), the first image is an effective atomic number image, and the processor outputs the effective atomic number image generated on the basis of the data to the display destination, and receives setting of the region on the effective atomic number image output to the display destination.

(10) The data processing apparatus according to any one of (1) to (9), in which the processor receives setting of a range of the second image that is the analysis target, and analyzes the second image within the set range to generate the statistical information.

(11) The data processing apparatus according to any one of (1) to (10), in which the processor outputs a screen for setting conditions for reconstruction in a case where the reconstruction is performed by changing the conditions, to the display destination, and displays the first image on the screen.

(12) The data processing apparatus according to any one of (1) to (10), in which the processor outputs a screen for setting conditions for imaging and reconstruction to the display destination, and displays the first image on the screen.

(13) The data processing apparatus according to any one of (1) to (12), in which the spectral CT device is a photon counting CT device.

(14) A data processing method of processing data of a plurality of slices acquired by a spectral CT device, the data processing method including a step of outputting a first image generated on the basis of the data to a display destination; a step of receiving setting of a region that is an analysis target, on the first image output to the display destination; a step of analyzing a second image of the plurality of slices generated on the basis of the data to generate statistical information indicating a distribution of a material in the region for each slice; and a step of outputting the statistical information to the display destination.

(15) A data processing program for processing data of a plurality of slices acquired by a spectral CT device, the data processing program causing a computer to execute a function of outputting a first image generated on the basis of the data to a display destination; a function of receiving setting of a region that is an analysis target, on the first image output to the display destination; a function of analyzing a second image of the plurality of slices generated on the basis of the data to generate statistical information indicating a distribution of a material in the region for each slice; and a function of outputting the statistical information to the display destination.

According to the present invention, it is possible to improve accuracy of image selection.

### BRIEF DESCRIPTION OF THE DRAWINGS

FIG. 1 is a schematic configuration diagram of a PCCT device.

FIG. 2 is a diagram illustrating an example of a hardware configuration of a console.

FIG. 3 is a block diagram of main functions of a console regarding generation of a tomographic image.

FIG. 4 is a block diagram of main functions of a console regarding an analysis function.

FIG. 5 is a diagram illustrating an example of a screen for receiving setting of an analysis target region.

FIG. 6 is a diagram illustrating an example of setting of an analysis target region by using a region setting frame.

FIG. 7 is a diagram illustrating an example of a histogram.

FIG. 8 is a diagram illustrating an example of display of an analysis result.

FIG. 9 is a flowchart illustrating an operation procedure of processing of obtaining a distribution of a density value of a material.

FIG. 10 is a diagram illustrating another example of display of an analysis result.

FIG. 11 is a diagram illustrating an example of a case where a histogram is generated by designating a range of density values to be displayed.

FIG. 12 is a diagram illustrating another example of display of a histogram.

FIG. 13 is a diagram illustrating an example of a post-reconstruction setting screen that enables checking of a distribution of density values for each slice.

FIG. 14 is a diagram illustrating another example of display of an analysis result.

FIG. 15 is a diagram illustrating an example of an imaging plan screen that enables checking of a distribution of density values for each slice.

FIG. 16 is a diagram illustrating another example of display of an analysis result.

FIG. 17 is a block diagram of main functions of a console regarding an analysis function.

FIG. 18 is a diagram illustrating an example of a histogram.

FIG. 19 is a flowchart illustrating an operation procedure of processing of obtaining a distribution of an effective atomic number.

FIG. 20 is a diagram illustrating an example of a histogram displayed in an emphasized manner.

FIG. 21 is a diagram illustrating an example of a setting method of a threshold value.

### DESCRIPTION OF THE PREFERRED EMBODIMENTS

Hereinafter, preferred embodiments for carrying out the present invention will be described with reference to the accompanying drawings.

### First Embodiment

Here, a case where the present invention is applied to an X-ray computed tomography device (PCCT device) capable of performing photon counting computed tomography (PCCT) will be described as an example.

### PCCT Device

The PCCT device is an X-ray CT device that employs a photon counting type X-ray detection device for detecting X-rays. Detection data obtained by the PCCT device enables spectral imaging, and enables the generation (reconstruction) of images with various kinds of information, such as virtual monochromatic X-ray images, effective atomic number images, and material discrimination images.

Here, the virtual monochromatic X-ray image is an image obtained by virtually expressing an image obtained at a single energy.

The effective atomic number image is an image illustrating a distribution of an effective atomic number (effective Z) of a material. The effective atomic number is an atomic number corresponding to constituent elements of a compound or a mixture when viewed in an averaged manner. In the effective atomic number image, the effective atomic number is represented for each pixel.

The material discrimination image is an image representing a distribution of density values of a material. In the material discrimination image, a density value of a material is represented for each pixel. The material discrimination image is also referred to as a material density image.

The PCCT device is an example of a spectral CT device.

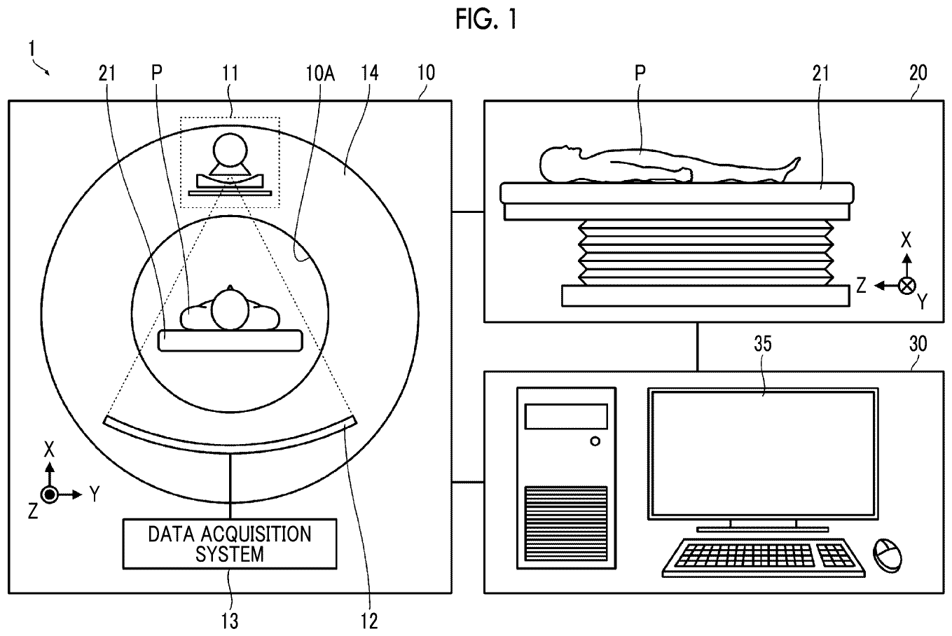

FIG. 1 is a schematic configuration diagram of a PCCT device. Note that in FIG. 1, an X axis, a Y axis, and a Z axis are three axes orthogonal to each other. A Y-axis direction and a Z-axis direction are defined as a horizontal direction, and an X-axis direction is defined as a vertical direction (up-down direction). In addition, the Z-axis direction is defined as a body axis direction.

As illustrated in FIG. 1, a PCCT device 1 includes a scanner gantry 10, an examination table 20, a console 30, and the like. The respective apparatuses are connected to each other in a communicable manner.

### Scanner Gantry

The scanner gantry 10 has an opening portion (bore), and irradiates a subject P inserted into an opening portion 10A with X-rays to execute the scan of the PCCT. The scanner gantry 10 includes an X-ray tube device 11, an X-ray detection device 12, a data acquisition system (DAS) 13, a rotation frame 14, and the like.

The X-ray tube device 11 irradiates the subject P with X-rays. The X-ray tube device 11 includes an X-ray tube, an X-ray high-voltage device, a bowtie filter, a collimator, and the like. The X-ray tube, which is an X-ray source, outputs X-rays by applying a high voltage from the X-ray high-voltage device. The subject P is irradiated with the X-rays output from the X-ray tube through the bowtie filter and the collimator.

The X-ray detection device 12 detects the X-rays emitted from the X-ray tube device 11 and transmitted through the subject P. The X-ray detection device 12 is a photon counting type X-ray detection device. The photon counting type X-ray detection device outputs an electric signal corresponding to the number of photons as a detection signal of X-rays. The X-ray detection device 12 has, for example, a structure in which a plurality of detection elements are two-dimensionally arranged in a channel direction (circumferential direction) and a column direction (body axis direction).

The data acquisition system 13 collects the electric signals output from each detection element of the X-ray detection device 12, and generates detection data. The detection data is data in which a count value (count number) of X-ray photons is assigned for each energy bin. The energy bin is a section in which the X-ray spectrum is divided into energy bandwidths. The detection data generated by the data acquisition system 13 is output to the console 30.

The rotation frame 14 has a cylindrical shape and is rotated around an axis by being driven by a rotary drive unit (not illustrated). An inner peripheral portion of the rotation frame 14 constitutes the opening portion 10A of the scanner gantry 10. The X-ray tube device 11 and the X-ray detection device 12 are mounted on the rotation frame 14. The X-ray tube device 11 and the X-ray detection device 12 are disposed to face each other with the opening portion 10A interposed therebetween. By rotating the rotation frame 14, the X-ray tube device 11 and the X-ray detection device 12 are rotated around a rotation axis of the rotation frame 14. The rotation axis of the rotation frame 14 constitutes an imaging center.

### Examination Table

The subject P is placed on the examination table 20, and the examination table 20 is moved in both the up-down direction and the horizontal direction. The examination table 20 includes a top plate 21 on which the subject P is placed. The top plate 21 is driven by an up-down drive unit (not illustrated), and is moved up and down in the vertical direction. In addition, the top plate 21 is driven by a horizontal drive unit (not illustrated), and is moved horizontally in the body axis direction (Z-axis direction). The position (height) of the subject P in the up-down direction is adjusted by moving the top plate 21 up and down. In addition, the subject P is moved in the opening portion 10A of the scanner gantry 10 along the body axis direction by moving the top plate 21 horizontally along the body axis direction.

### Console

The console 30 functions as an operation panel, and also functions as a data processing apparatus that performs predetermined image processing and analysis processing.

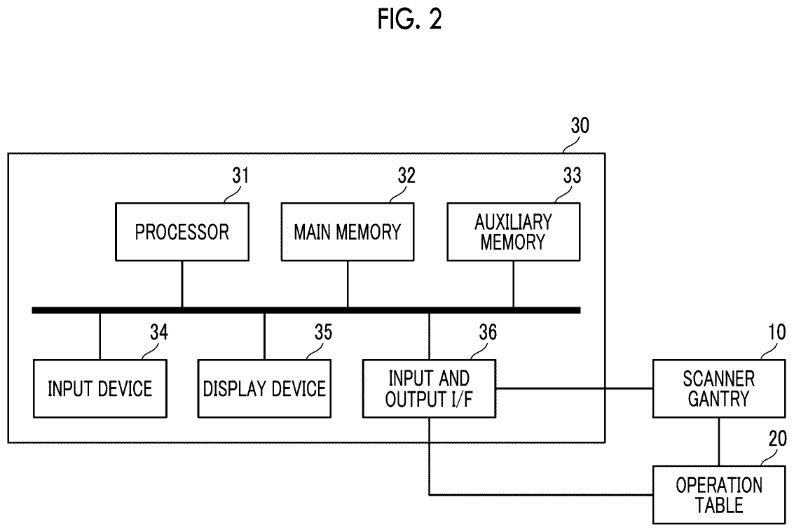

FIG. 2 is a diagram illustrating an example of a hardware configuration of the console.

The console 30 is configured by a computer, and includes a processor 31, a main memory 32, an auxiliary memory 33, an input device 34, a display device 35, an input and output interface 36, and the like.

As the processor 31, for example, a central processing unit (CPU) which is a general-purpose processor that executes a program to function as various processing units is adopted. Various programs executed by the processor 31 and data are stored in the main memory 32 and/or the auxiliary memory 33. The program is synonymous with software.

The main memory 32 includes a random access memory (RAM) and a read only memory (ROM).

The auxiliary memory 33 is configured of, for example, a hard disk drive (HDD) or a solid state drive (SSD).

The input device 34 includes, for example, a keyboard, a mouse, a touch panel, and the like.

The display device 35 is configured of, for example, a liquid crystal display (LCD) or an organic electro luminescence diode display (OLED display). In the present embodiment, the display device 35 is an example of a display destination.

The input and output interface (I/F) 36 communicably connects the console 30 to the scanner gantry 10 and the examination table 20.

### Function as Operation Panel

The console 30 integrally controls the overall operation of the PCCT device 1 on the basis of an operation input from a user. The console 30 receives an operation input from the user by using the input device 34 and the display device 35 as a user interface. An imaging execution instruction, setting of conditions for imaging and reconstruction, setting of conditions for reconstruction in post-reconstruction, setting of analysis conditions, and the like are performed via the console 30. Note that the post-reconstruction refers to processing in which reconstruction is performed again after the imaging, after setting different conditions from those during the imaging (post-reconstruction).

### Function as Data Processing Apparatus

### (a) Function of Generating Image (Image Processing Function)

The console 30 has a function of processing the detection data of a plurality of slices obtained by imaging and of generating a predetermined tomographic image (slice image). The generatable tomographic images include a virtual monochromatic X-ray image, an effective atomic number image, a material discrimination image, and the like, in addition to a normal tomographic image illustrating a distribution of a linear attenuation coefficient.

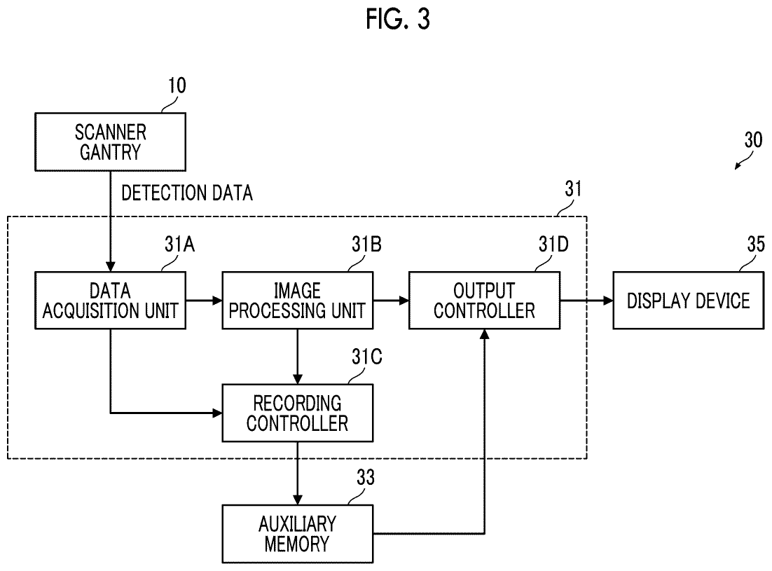

FIG. 3 is a block diagram of main functions of the console regarding generation of the tomographic image.

As illustrated in FIG. 3, regarding the generation of the tomographic image, the console 30 has functions of a data acquisition unit 31A, an image processing unit 31B, a recording controller 31C, an output controller 31D, and the like. The functions of the respective units are realized by the processor 31 executing a predetermined program.

The data acquisition unit 31A acquires detection data of X-rays from the scanner gantry 10. As described above, the detection data is data in which the count value of the X-ray photon is assigned for each energy bin. The detection data includes information on a channel number and a column number of the detection element, a view number indicating a collected view, and a count value for each energy bin of the detected X-ray photon.

The image processing unit 31B performs predetermined reconstruction processing on the detection data acquired by the data acquisition unit 31A to generate a tomographic image. The tomographic image generated by the image processing unit 31B includes a virtual monochromatic X-ray image, an effective atomic number image, a material discrimination image, and the like, in addition to a normal tomographic image.

The recording controller 31C records the image generated by the image processing unit 31B, in the auxiliary memory 33. The image is recorded in an examination unit. Detection data of a generation source is recorded in association with the image of each examination. As a result, post-reconstruction is possible. Each image is assigned a slice number in an imaging order, and is recorded in an identifiable manner.

The output controller 31D outputs the tomographic image generated by the image processing unit 31B to the display device 35. In addition, the output controller 31D outputs the recorded tomographic image to the display device 35.

### (b) Analysis Function

The console 30 according to the present embodiment has a function of generating a graph illustrating a distribution of a material (frequency distribution of a presence amount of each material) in a designated region for each slice and presenting the graph to the user (analysis function), as a function of supporting the interpretation. More specifically, a graph illustrating a distribution of density values of a material (frequency distribution of a presence amount of each density value) is generated for each slice, and is presented to the user.

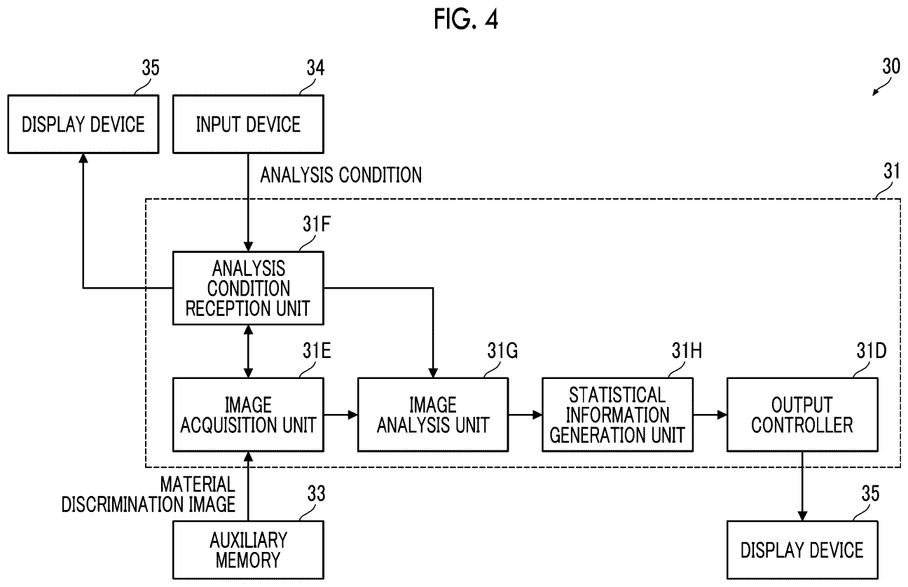

FIG. 4 is a block diagram of main functions of the console regarding the analysis function.

As illustrated in FIG. 4, regarding the analysis function, the console 30 has functions of an image acquisition unit 31E, an analysis condition reception unit 31F, an image analysis unit 31G, a statistical information generation unit 31H, the output controller 31D, and the like. The functions of the respective units are realized by the processor 31 executing a predetermined program. The program is an example of a data processing program.

### (a) Image Acquisition Unit

The image acquisition unit 31E acquires an image (slice image) that is an analysis target. In the present embodiment, the image that is the analysis target is a material discrimination image. The image acquisition unit 31E acquires a series of material discrimination images obtained in one examination, as the image that is the analysis target. The image acquisition unit 31E reads out and acquires the series of material discrimination images of the examination designated by the user, from the auxiliary memory 33. The selection of the examination (image group) as the analysis target is performed on a predetermined selection screen. The user selects the examination (image group) as the analysis target, on the predetermined selection screen. In the present embodiment, the material discrimination image is an example of a second image.

### (b) Analysis Condition Reception Unit

The analysis condition reception unit 31F receives the setting of a region (analysis target region) that is the analysis target, as the analysis condition. The setting of the analysis target region is performed on the material discrimination image displayed on the screen by outputting the material discrimination image to the display device 35. The analysis target region is substantially synonymous with a region of interest (ROI).

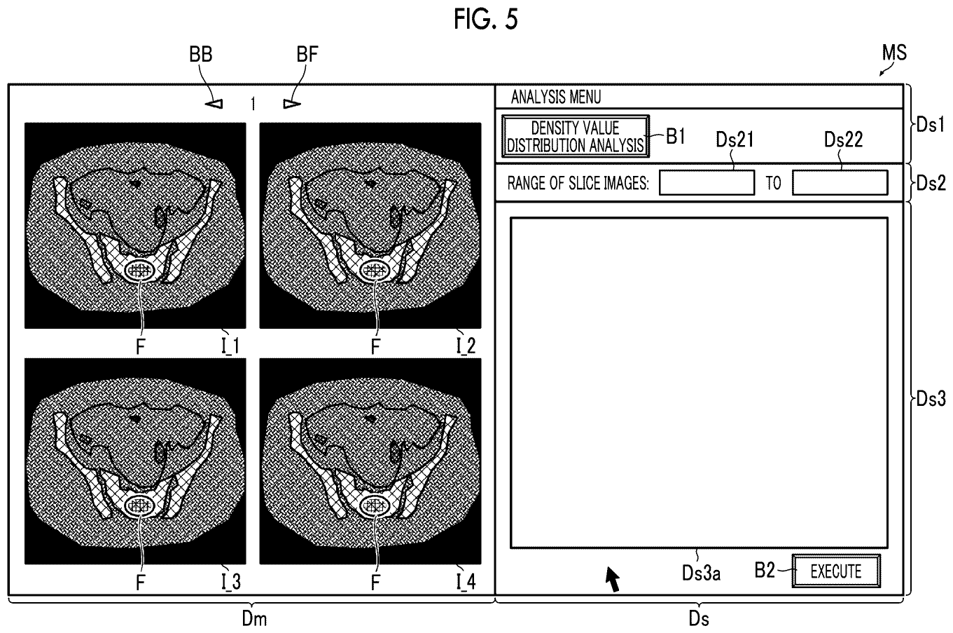

FIG. 5 is a diagram illustrating an example of a screen for receiving the setting of the analysis target region.

As illustrated in FIG. 5, a main display region Dm and a sub-display region Ds are set on a screen MS.

In the main display region Dm, material discrimination images I_n (n=1, 2, . . . ) as the analysis targets are displayed. In the example illustrated in FIG. 5, an example of a case where four images are displayed at one time is illustrated. The number of images to be displayed at one time is not limited thereto. At least one image may be displayed. The image displayed in the main display region Dm can be switched by clicking an image forward button Bf or an image backward button Bb. The images are displayed in an order of slice numbers, and are switched in the order of slice numbers.

An analysis menu display region Ds1, an image range designation region Ds2, and an analysis result display region Ds3 are set in the sub-display region Ds.

Buttons for the analysis processing that can be executed on the image being displayed in the main display region Dm are displayed in the analysis menu display region Ds1. In the PCCT device 1 of the present embodiment, since the analysis of the distribution of the density values is possible, at least a button (density value distribution analysis button) B1 of the function is displayed in the analysis menu display region Ds1. In a case of performing the analysis processing of the distribution of the density value, the user clicks a density value distribution analysis button B1 displayed in the analysis menu display region Ds1.

In a case where the density value distribution analysis button B1 is clicked, a frame (region setting frame) F is displayed to be superimposed on the material discrimination images I_n being displayed in the main display region Dm. The user sets the analysis target region by using the region setting frame F.

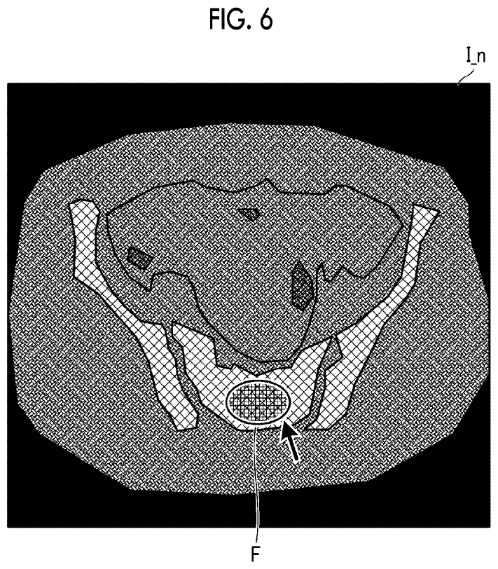

FIG. 6 is a diagram illustrating an example of the setting of the analysis target region by using the region setting frame.

For example, as an initial display, the region setting frame F is displayed in a predetermined size circle and is displayed at a center position of the image. The user sets the analysis target region at a desired position by adjusting the position, the size, and the shape (the aspect ratio of an ellipse) of the region setting frame F displayed in a superimposed manner on the material discrimination image I_n. The adjustment of the position and the like is performed by, for example, a mouse operation.

The region setting frame F is displayed on all of the material discrimination images I_n displayed in the main display region Dm. The adjustment can be performed on all of the images, and the adjustment performed on one image is also reflected on the other images.

The image range designation region Ds2 is a region for designating a range of slice images (material discrimination images in the present embodiment) that are the analysis targets. As illustrated in FIG. 5, the image range designation region Ds2 includes a box Ds21 for inputting a start point of the range of the slice images that are the analysis targets, and a box Ds22 for inputting an end point of the range. In a case of performing the analysis by narrowing down the range, the user inputs the start point of the range to the box Ds21 on one side (left side in the drawing), and inputs the end point of the range to the box Ds22 on the other side (right side in the drawing). For example, in a case where the number of slice images acquired by the image acquisition unit 31E is 60, in a case where the 20th to 35th slice images are the analysis targets, “20” is input to the box Ds21 on one side (left side in the drawing), and “35” is input to the box Ds22 on the other side (right side in the drawing). In a case where the number of slice images as the analysis target is large, it takes time for the analysis processing. In a case where the range of the slice images to be analyzed is narrowed to some extent, the analysis is executed by narrowing down the range. As a result, the processing time required for the analysis can be shortened.

The analysis result display region Ds3 is a region where the analysis result is displayed. The display of the analysis result will be described below. As illustrated in FIG. 5, an execution button B2 is displayed in the analysis result display region Ds3. The execution button B2 is a button for an instruction to execute the analysis. After the analysis target region is set, the user clicks the execution button B2 to give an instruction to execute the analysis.

### (c) Image Analysis Unit

The image analysis unit 31G individually analyzes the material discrimination image as the analysis target, and obtains a density value of the material present in the analysis target region and an area thereof (area occupied by the density value of each material in the analysis target region). The area is calculated on the basis of an image resolution (pixel/mm). The analysis is performed on all of the material discrimination images acquired by the image acquisition unit 31E.

### (d) Statistical Information Generation Unit

The statistical information generation unit 31H generates statistical information on the basis of the analysis result by the image analysis unit 31G. More specifically, a graph illustrating the distribution of density values of the material (frequency distribution of an area for each density value) for each slice is generated as the statistical information. As an example, in the present embodiment, a bivariate histogram illustrating the distribution of the density values of the material for each slice is generated.

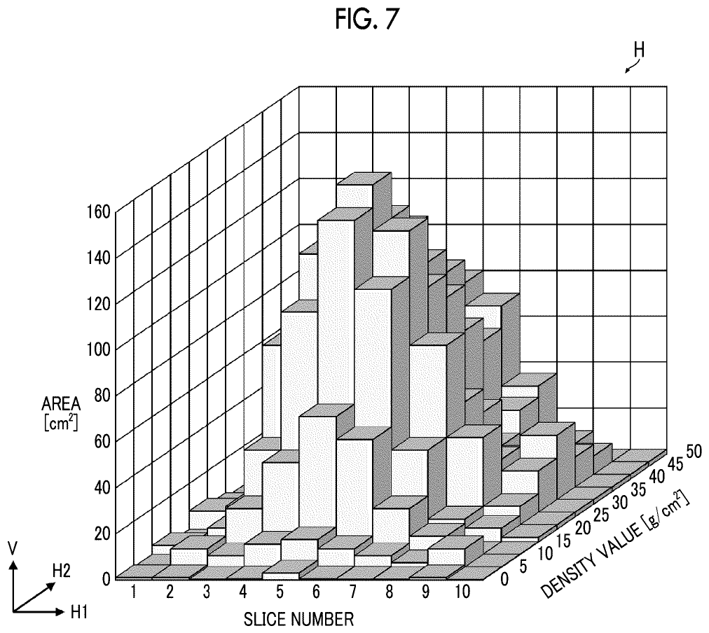

FIG. 7 is a diagram illustrating an example of the histogram.

As illustrated in FIG. 7, a bivariate histogram H in which a first horizontal axis H1 is the slice number, a second horizontal axis H2 is the density value of the material, and a vertical axis V is the area is generated. The histogram H corresponds to a display in which histograms of each slice (histograms representing the distribution of the presence amount of the density value of the material in the analysis target region of each slice) are superimposed in the order of slice numbers. As illustrated in FIG. 7, the histogram H is generated as a three-dimensional graph.

### (e) Output Controller

The output controller 31D outputs, as the analysis result, the histogram H generated by the statistical information generation unit 31H to the display device 35. As described above, the analysis result is displayed in the analysis result display region Ds3.

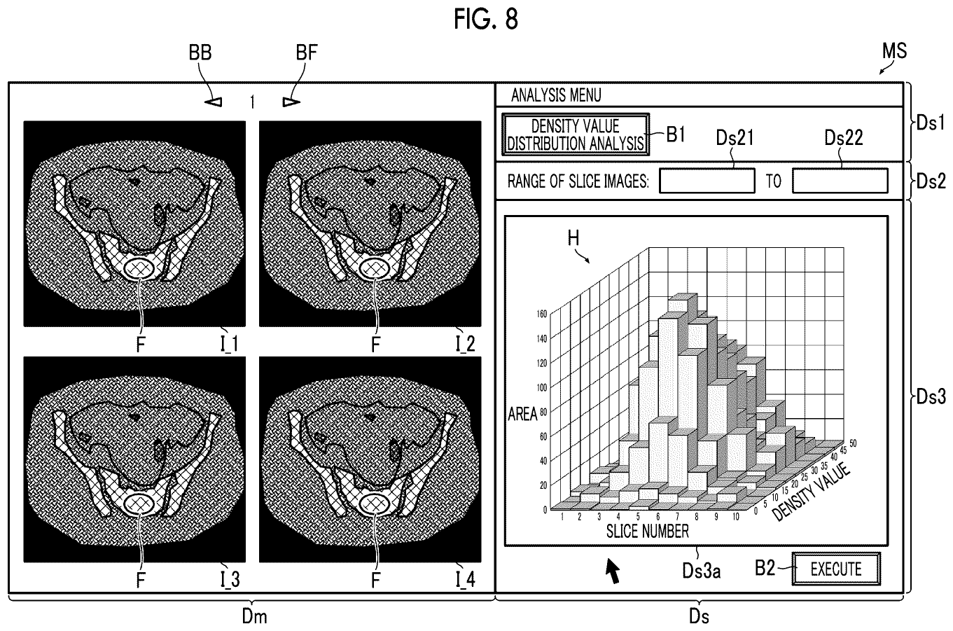

FIG. 8 is a diagram illustrating an example of display of the analysis result.

As illustrated in FIG. 8, the histogram H as the analysis result is displayed in a graph display region Ds3a set in the analysis result display region Ds3.

The histogram H displayed in the graph display region Ds3a is displayed by being enlarged or reduced by an enlargement or reduction operation by the user. In addition, the display position is changed by a movement operation by the user. In addition, the viewpoint is changed (the displayed orientation is changed) by a rotation operation by the user. The operation for the enlargement, reduction, or the like is performed by, for example, a mouse.

### Processing Operation of Analysis

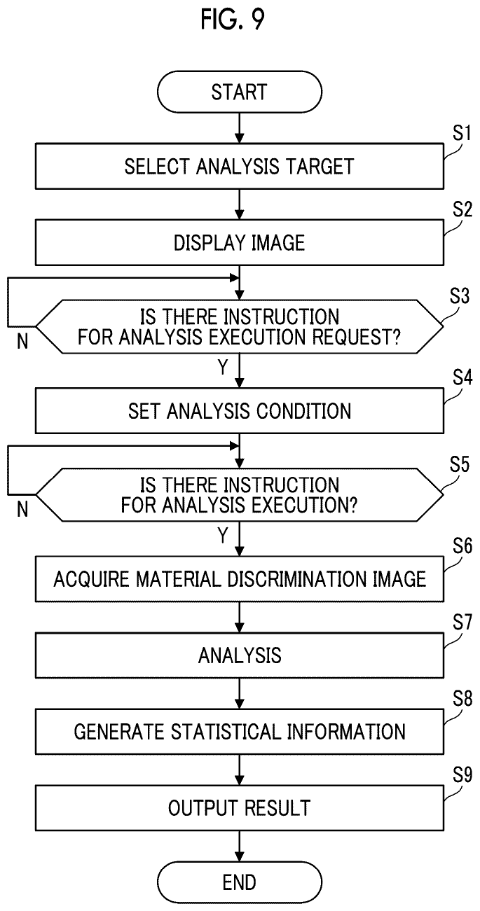

FIG. 9 is a flowchart illustrating an operation procedure of processing of obtaining the distribution of the density values of the material.

First, an analysis target is selected (step S1). As described above, the analysis is performed on a series of material discrimination images obtained in one examination. The user designates the examination as the analysis target, and selects an image group that is the analysis target. The selection of the examination as the analysis target is performed on a predetermined selection screen.

In a case where the analysis target is selected, the image is displayed (step S2). Specifically, the material discrimination image obtained by the examination as the analysis target is output to the display device 35, and is displayed in the main display region Dm in the screen MS (refer to FIG. 5).

Next, it is determined whether or not there is an execution request of the analysis (step S3). The execution request of the analysis is made by clicking the density value distribution analysis button B1. The processor 31 determines whether or not there is an execution request of the analysis by determining whether or not the density value distribution analysis button B1 is clicked.

In a case where the execution request of the analysis is received, the analysis condition is set (step S4). The processor 31 displays the region setting frame F, in a superimposed manner, on the material discrimination image I_n displayed in the main display region Dm to receive the setting of the analysis target region from the user (refer to FIGS. 5 and 6). The user sets the analysis target region by adjusting the position, the size, and the shape (aspect ratio) of the region setting frame F displayed in a superimposed manner on the material discrimination image I_n.

In addition, the user sets the range of the material discrimination images as the analysis targets, as necessary. In a case of performing the analysis by narrowing down the range, the range as the analysis target is set in the image range designation region Ds2.

After the setting of the analysis target region is completed, the user clicks the execution button B2 displayed in the sub-display region Ds in the screen MS to give an instruction to execute the analysis (refer to FIG. 5). The processor 31 determines whether or not there is an instruction to execute the analysis by determining whether or not the execution button B2 is clicked (step S5).

In a case where an instruction to execute the analysis is given, the material discrimination image as the analysis target is acquired (step S6). More specifically, a series of material discrimination images as the analysis target is read out from the auxiliary memory 33. Then, the analysis processing is performed on the acquired series of material discrimination images (step S7). Specifically, the density value of the material present in the analysis target region and the area thereof are obtained for each image.

In a case where the analysis of all the images is completed, the statistical information is generated on the basis of the analysis result (step S8). Specifically, a graph illustrating the distribution of the density values of the material (frequency distribution of the areas for each density value) is generated for each slice. In the present embodiment, the bivariate histogram H illustrating the distribution of the density values of the material for each slice is generated (refer to FIG. 7).

The generated histogram H is output, as the analysis result, to the display device 35 (step S9). In the present embodiment, as illustrated in FIG. 8, the histogram H is displayed on the same screen as the screen on which the material discrimination image I_n as the analysis target is displayed.

As described above, the histogram H is composed of a three-dimensional graph in which the first horizontal axis H1 is the slice number, the second horizontal axis H2 is the density value of the material, and the vertical axis V is the area (refer to FIG. 7). By checking the histogram H, it is possible to easily ascertain a distribution status of the density value of the material for each slice. As a result, for example, it is possible to easily check which slice has the density value specific to the symptom. In addition, this improves the accuracy of image selection in the analysis.

### Modification Example

### Display of Analysis Result

In the above-described embodiment, as illustrated in FIG. 8, the histogram H of the analysis result is displayed on the same screen as the screen on which the material discrimination image I_n as the analysis target is displayed, but the display form of the analysis result is not limited thereto.

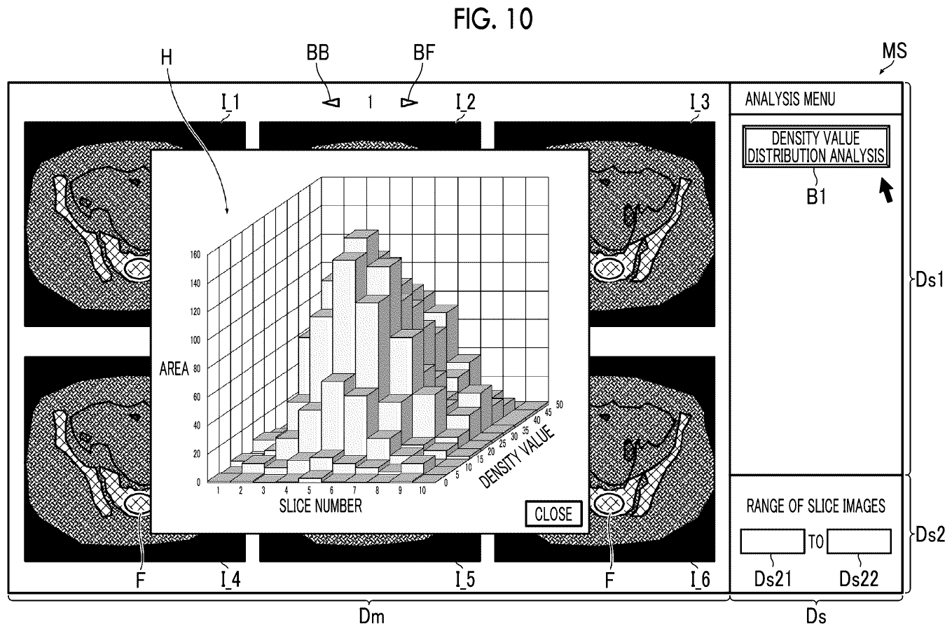

FIG. 10 is a diagram illustrating another example of the display of the analysis result.

FIG. 10 illustrates an example of a case where the histogram H of the analysis result is displayed in a pop-up manner on a separate screen (separate window).

In addition, for example, a configuration may be adopted in which only the histogram H is displayed by switching the screen.

### Designation of Display Range

In the histogram to be displayed as the statistical information, a configuration may be adopted in which the user can arbitrarily designate a range of the density values to be displayed.

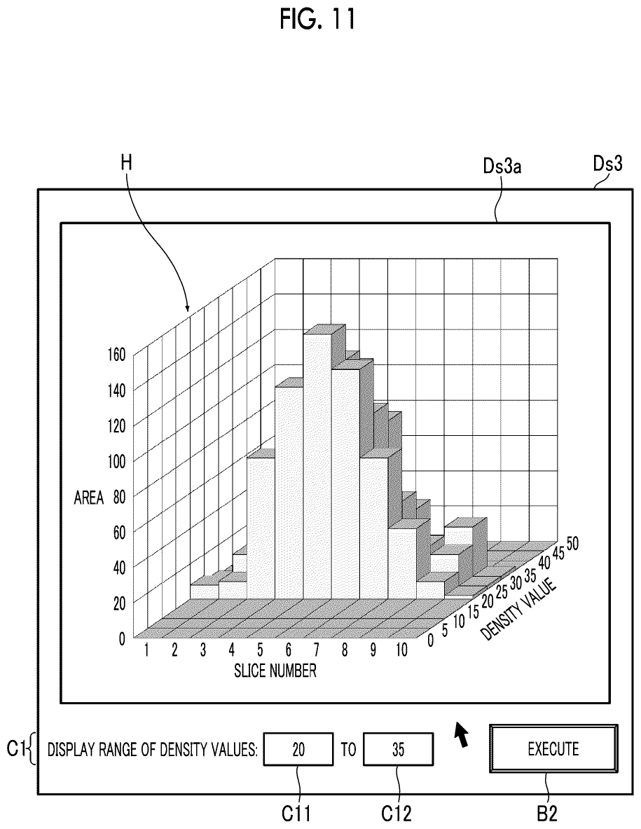

FIG. 11 is a diagram illustrating an example of a case where the histogram is generated by designating a range of density values to be displayed.

As illustrated in FIG. 11, a field (display range setting field) C1 for setting a display range of the density values is provided in the analysis result display region Ds3. The display range setting field C1 includes a box C11 for inputting a start point of the display range and a box C12 for inputting an end point of the display range.

In a case where the histogram H is displayed by designating the display range, the user inputs the density value of the desired display range in the display range setting field C1, and clicks the execution button B2.

In a case where the display range is designated, the histogram H is generated in the designated display range and is displayed in the graph display region Ds3a. The histogram H illustrated in FIG. 11 illustrates an example of a case where the distribution is displayed only in a range (20 to 35 [g/cm²]) designated by the user with respect to the histogram H of the example illustrated in FIG. 8.

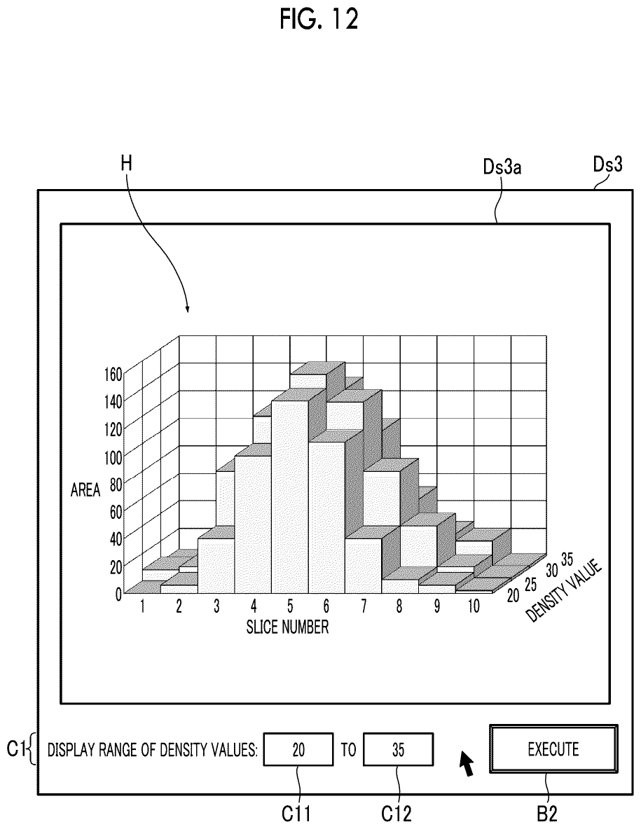

FIG. 12 is a diagram illustrating another example of the display of the histogram.

The example illustrated in FIG. 12 illustrates an example of a case of generating and displaying the histogram H in a range (20 to 35 [g/cm²]) of the density values designated by the user. That is, an example of a case of generating and displaying the histogram H by limiting a range of the second horizontal axis indicating the density value of the material to a range designated by the user is illustrated.

By generating and displaying the histogram H by limiting the display range of the density value in this manner, it is possible to easily check the distribution of the density values in a specific range. As a result, it is possible to more easily check which slice has the density value unique to the symptom.

Note that in the above example, the histogram is generated to display only the range of the density value designated by the user, but for example, a histogram in which the display of the range of the density value designated by the user is changed while the entire distribution is displayed may be generated and displayed. That is, a histogram in which a range of the density value designated by the user is displayed in an emphasized manner or the like and which is displayed in a form distinguishable from others is generated. For example, a histogram in which a range of the designated density value is distinguishable from others can be generated by displaying the range of the designated density value in a color different from other colors.

### Provision of Analysis Function on Setting Screen of Post-Reconstruction

In a case of performing post-reconstruction, post-reconstruction may be performed by limiting the range to a part of the image range. For example, the post-reconstruction may be performed by limiting the range to an image range in which a medical case is present. In this case, the user needs to perform the post-reconstruction by designating the image range.

In general, in the post-reconstruction processing, setting of various conditions is performed on a dedicated setting screen (post-reconstruction setting screen). In a case where the post-reconstruction is performed by limiting the range to an image range in which a medical case is present, the designation of the image range can be easily performed in a case where the distribution of the density values for each slice can be checked on the post-reconstruction setting screen.

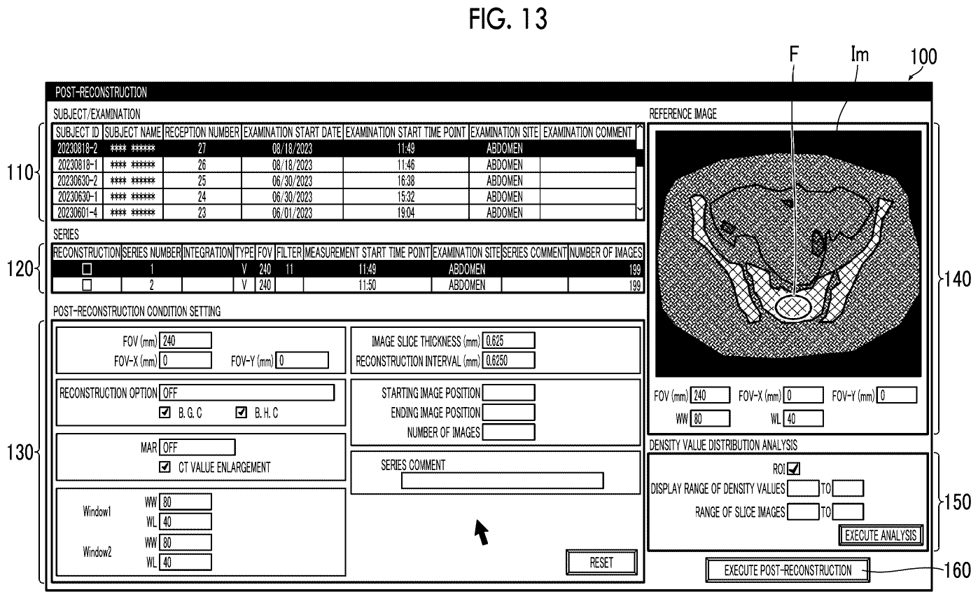

FIG. 13 is a diagram illustrating an example of the post-reconstruction setting screen that enables checking of the distribution of the density values for each slice.

As illustrated in FIG. 13, a post-reconstruction setting screen 100 of the present example includes a subject information display region 110, a series information display region 120, a post-reconstruction condition setting region 130, a reference image display region 140, an analysis condition setting region 150, a post-reconstruction execution button 160, and the like.

In the subject information display region 110, information on the examined (imaged) subject is displayed in a list format. The information displayed in the subject information display region 110 includes a subject identification (ID), a subject name, a reception number, an examination start date, an examination start time point, an examination site, an examination comment, and the like. A subject to be a target for the post-reconstruction is selected from the subjects displayed in the subject information display region 110.

In the series information display region 120, the information of the captured image group of the subject selected in the subject information display region 110 is displayed as the series information in a list format. The information that can be displayed in the series information display region 120 includes a series number (a number of a series given to a series of image groups), integration, type, a field of view (FOV), a filter, a measurement start time point, an examination site, a series comment, the number of images, and the like. The image group of the series selected from the series displayed in the series information display region 120 is the target of the post-reconstruction.

The post-reconstruction condition setting region 130 is a region for setting conditions for the reconstruction in a case of performing post-reconstruction. The post-reconstruction condition setting region 130 includes an input field for inputting various conditions (parameters) of the reconstruction. The settable conditions include an FOV size, an FOV center (FOV-X, FOV-Y), a reconstruction option, a bowel gas correction (B.G.C), a beam hardening correction (B.H.C), a metal artifact reduction (MAR), a window value (window wide (WW), window level (WL)), an image slice thickness, a reconstruction interval, a reconstruction range (starting image position, ending image position), the number of images, a series comment, and the like. As the initial display, the setting at the time of imaging (setting at the time of post-reconstruction in the case of the post-reconstructed image group) is displayed in each input field. The user inputs the conditions for the post-reconstruction by changing the numerical value of each displayed parameter to an optional value. In a case of performing the reconstruction by limiting the range to a part of the image range, the image range is designated in the field of the reconstruction range. That is, the range for the reconstruction is designated by designating the starting image position and the ending image position in the field of the reconstruction range.

In the reference image display region 140, one image in the image group (series) selected as the target of the post-reconstruction is displayed as a reference image Im. For example, the image of the starting slice is displayed as the reference image Im. It is preferable that the reference image Im is optionally switched. Note that the image displayed as the reference image Im is not limited to the material discrimination image, and may be another image.

The reference image display region 140 includes input fields for parameters such as an FOV size, an FOV center (FOV-X, FOV-Y), and window values (WW, WL). The condition at the time of imaging is displayed as the initial display in the input field of each parameter. In a case of changing the numerical value displayed in the input field of each parameter, the change is reflected in the reference image being displayed. That is, the preview of the reconstruction is performed. Therefore, the conditions for the post-reconstruction can be determined by adjusting each parameter while checking the reference image.

The analysis condition setting region 150 is a region for setting the analysis condition in a case of performing the distribution analysis of the density value of the material for each slice. The analysis condition setting region 150 includes a check box for turning on and off the setting of the ROI, a box for designating the display range of the density value, a box for designating the range of the slice image as the analysis target, an analysis execution button for giving an instruction to execute the analysis, and the like. In a case where a check box for turning on and off the setting of the ROI is checked, the frame (region setting frame) F is displayed on the reference image Im displayed in the reference image display region 140, and the setting of the ROI is enabled. The region set as the ROI by the region setting frame F is set as the analysis target region. In addition, in a case where the range (start point and end point) of the slice image is designated using the box for designating the range of the slice image as the analysis target, the slice images in the designated range are set as the analysis targets. The instruction to execute the analysis is given by inputting the analysis condition and clicking the analysis execution button.

In a case where the instruction to execute the analysis is given, the distribution analysis of the density value is performed on the image group selected as the target of the post-reconstruction under the set conditions.

The post-reconstruction execution button 160 is a button for giving an instruction to execute the post-reconstruction. In a case where the post-reconstruction execution button 160 is clicked, the reconstruction is performed under the conditions set in the post-reconstruction condition setting region 130.

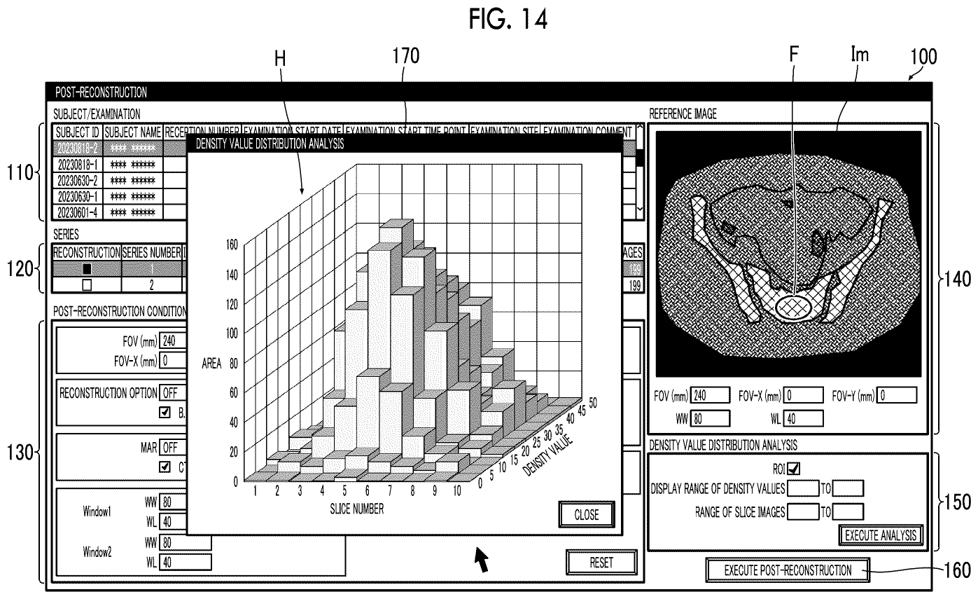

FIG. 14 is a diagram illustrating another example of the display of the analysis result.

As illustrated in FIG. 14, in a case where the distribution analysis of the density value is executed, a screen 170 of the analysis result is displayed in a pop-up manner. The user checks the histogram H displayed on the screen 170 of the analysis result, and determines the image range to be reconstructed by the post-reconstruction. That is, the image range is determined by checking which slice has the density value specific to the symptom.

As described above, by being able to check the distribution of the density value for each slice on the post-reconstruction setting screen 100, for example, it is possible to improve the accuracy of image selection in a case of performing the post-reconstruction only in the image range where the medical case is present.

In the present example, the post-reconstruction setting screen 100 is an example of a screen for setting the conditions for the reconstruction in a case of performing the reconstruction by changing the conditions. In addition, the reference image Im displayed in the reference image display region 140 is an example of a first image.

Note that in the above example, the analysis result is displayed on a separate screen (separate window), but a configuration may be adopted in which a display region for the analysis result is provided in the post-reconstruction setting screen 100 and the analysis result is displayed in the region.

### Provision of Analysis Function on Imaging Plan Screen

In the examination, the same subject may be imaged a plurality of times by changing the conditions for imaging and/or reconstruction. For example, in the examination of the abdomen, after the entire abdomen is imaged, the imaging may be performed again under different imaging conditions and/or reconstruction conditions, limited to a part where the medical case is present. In this case, the user needs to perform re-imaging by adjusting the imaging range in the body axis direction.

In general, the setting of the imaging conditions and the reconstruction conditions is performed on a dedicated setting screen (imaging plan screen). In a case of performing the imaging only in the range where the medical case is present, it is possible to easily designate the range as long as the distribution of the density value for each slice can be checked on the imaging plan screen.

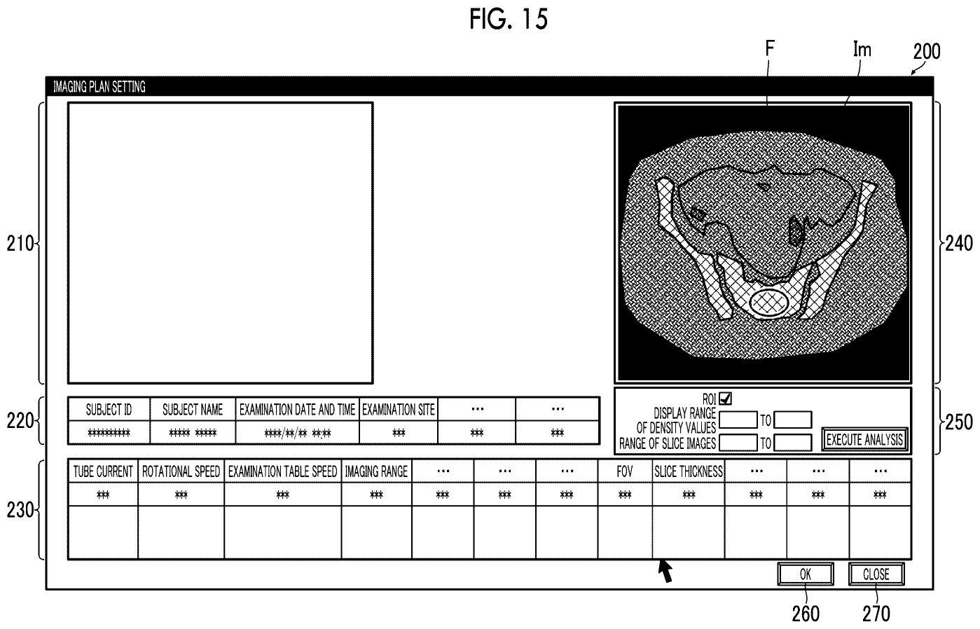

FIG. 15 is a diagram illustrating an example of the imaging plan screen that enables the checking of the distribution of the density values for each slice.

As illustrated in FIG. 15, an imaging plan screen 200 of the present example includes a scanogram display region 210, a subject information display region 220, an imaging condition setting region 230, an image display region 240, an analysis condition setting region 250, an OK button 260, a close button 270, and the like.

A scanogram is displayed in the scanogram display region 210. The scanogram is an image captured in advance in order to determine an imaging range.

The information on the subject is displayed in the subject information display region 220. The information displayed in the subject information display region 220 includes a subject identification (ID), a subject name, an examination date and time, an examination site, and the like.

The imaging condition setting region 230 is a region where conditions for imaging and reconstruction are set. The imaging condition setting region 230 includes input fields for inputting various conditions (parameters) for imaging and reconstruction. The user sets the conditions for imaging and reconstruction by inputting an appropriate numerical value in each displayed input field. The imaging range is set by inputting a numerical value for the imaging range in the input field for the imaging range.

One image of the image group (captured image group) obtained by imaging is displayed as the reference image Im in the image display region 240. For example, the image of the starting slice is displayed as the reference image Im. It is preferable that the reference image Im is optionally switched. Note that the image displayed as the reference image Im is not limited to the material discrimination image, and may be another image.

The analysis condition setting region 250 is a region for setting the analysis conditions in a case of performing a distribution analysis of the density value of the material for each slice. The analysis condition setting region 250 includes a check box for turning on and off the setting of the ROI, a box for designating the display range of the density value, a box for designating the range of the slice image as the analysis target, an analysis execution button for giving an instruction to execute the analysis, and the like. In a case where a check box for turning on and off the setting of the ROI is checked, the frame (region setting frame) F is displayed on the reference image Im displayed in the image display region 240, and the setting of the ROI is enabled. The region set as the ROI by the region setting frame F is set as the analysis target region. In addition, in a case where the range (start point and end point) of the slice image is designated using the box for designating the range of the slice image as the analysis target, the slice images in the designated range are set as the analysis targets. The instruction to execute the analysis is given by inputting the analysis condition and clicking the analysis execution button.

In a case where the instruction to execute the analysis is given, the distribution analysis of the density value is performed on the captured image group under the set conditions.

The OK button 260 is a button for giving an instruction to reflect the setting input as the conditions for imaging and reconstruction. The close button 270 is a button for giving an instruction to close the imaging plan screen 200.

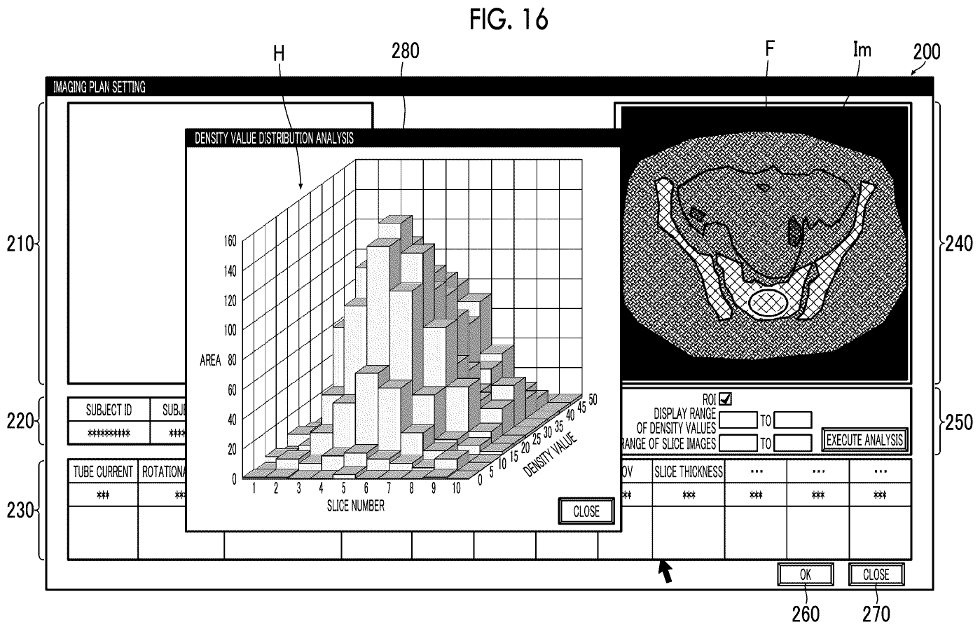

FIG. 16 is a diagram illustrating another example of the display of the analysis result.

As illustrated in FIG. 16, in a case where the distribution analysis of the density value is executed, a screen 280 of the analysis result is displayed in a pop-up manner. The user checks the histogram H displayed on the screen 280 of the analysis result to determine the imaging range. That is, the imaging range is determined by checking which slice has the density value specific to the symptom.

As described above, by being able to check the distribution of the density value for each slice on the imaging plan screen 200, for example, it is possible to improve the accuracy of image selection in a case of performing the imaging only in the range where the medical case is present.

In the present example, the imaging plan screen 200 is an example of a screen for setting the conditions for imaging and reconstruction. In addition, the reference image Im displayed in the image display region 240 is an example of the second image.

Note that in the above example, the analysis result is displayed on a separate screen (separate window), but a configuration may be adopted in which a display region for the analysis result is provided in the imaging plan screen 200 and the analysis result is displayed in the region.

### Second Embodiment

In the first embodiment, it is configured to analyze the effective atomic number image obtained by imaging and obtain the distribution of the material in the specific region based on the density value of the material. In the present embodiment, the effective atomic number image obtained by imaging is analyzed and the distribution of the material in the specific region is obtained based on the effective atomic number.

Note that the PCCT device 1 is the same as the PCCT device 1 of the first embodiment except for the difference in the analysis function, and therefore, only the analysis function will be described here.

### Analysis Function

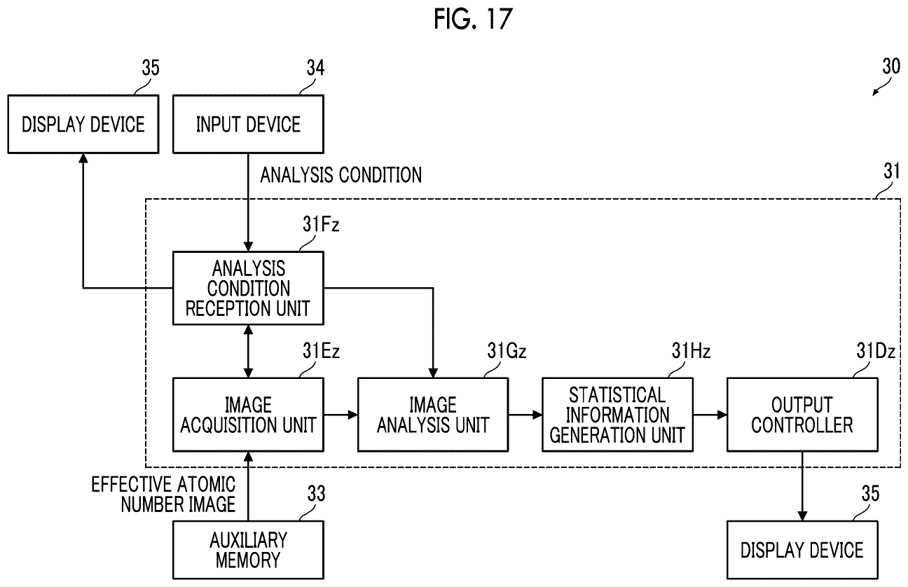

FIG. 17 is a block diagram of main functions of the console regarding the analysis function.

As illustrated in FIG. 17, regarding the analysis function, the console 30 has functions of an image acquisition unit 31Ez, an analysis condition reception unit 31Fz, an image analysis unit 31Gz, a statistical information generation unit 31Hz, the output controller 31Dz, and the like. The functions of the respective units are realized by the processor 31 executing a predetermined program. The program is an example of a data processing program.

### (a) Image Acquisition Unit

The image acquisition unit 31Ez acquires the image that is the analysis target. In the present embodiment, the image that is the analysis target is an effective atomic number image. The image acquisition unit 31Ez acquires a series of effective atomic number images obtained in one examination, as the image that is the analysis target. The image acquisition unit 31Ez reads out and acquires a series of effective atomic number images of the examination (series) designated by the user, from the auxiliary memory 33. In the present embodiment, the effective atomic number image is an example of the second image.

### (b) Analysis Condition Reception Unit

The analysis condition reception unit 31Fz receives the setting of the analysis target region as the analysis condition. The analysis condition reception unit 31Fz outputs the effective atomic number image to the display device 35, and accepts the setting of the analysis target region on the effective atomic number image displayed on the screen. Except for the difference in the type of image to be displayed, the unit is the same as that in the first embodiment described above. That is, the analysis target region is set by adjusting the position, size, and shape of the frame (region setting frame) displayed on the image (refer to FIGS. 6 and 7).

### (c) Image Analysis Unit

The image analysis unit 31Gz individually analyzes the effective atomic number image as the analysis target, and measures the presence amount of each effective atomic number in the analysis target region. Specifically, the number of pixels is counted for each effective atomic number by targeting the analysis target region of each image. The analysis is performed on all of the effective atomic number images acquired by the image acquisition unit 31Ez.

### (d) Statistical Information Generation Unit

The statistical information generation unit 31Hz generates statistical information on the basis of the analysis result by the image analysis unit 31Gz. More specifically, a graph illustrating the distribution of the effective atomic number (frequency distribution of the presence amount for each effective atomic number) for each slice is generated as the statistical information. As an example, in the present embodiment, a bivariate histogram illustrating the distribution of the effective atomic number for each slice is generated.

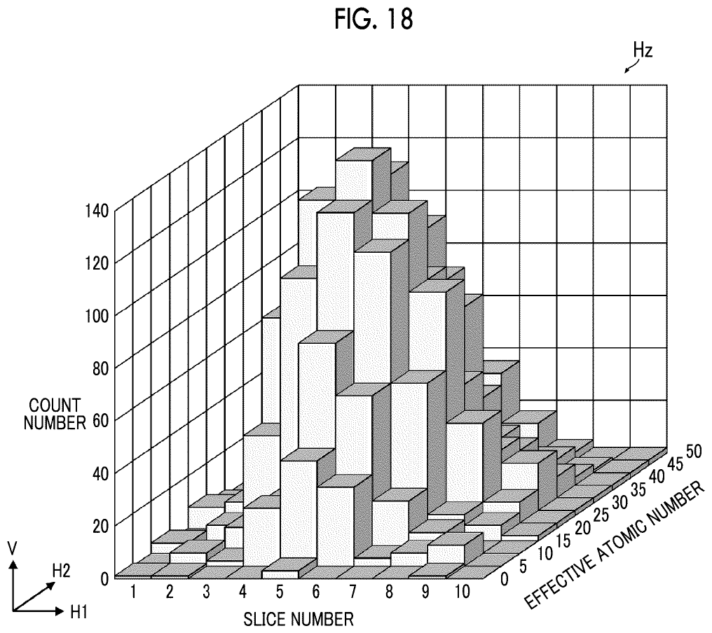

FIG. 18 is a diagram illustrating an example of the histogram.

As illustrated in FIG. 18, a bivariate histogram Hz in which the first horizontal axis H1 is the slice number, the second horizontal axis H2 is the effective atomic number, and the vertical axis V is the count number (presence amount) of the pixel is generated. The histogram Hz corresponds to a display in which histograms of each slice (histograms representing the distribution of each effective atomic number in the analysis target region of each slice) are superimposed in the order of slice numbers. As illustrated in FIG. 18, the histogram Hz is generated as a three-dimensional graph.

### (e) Output Controller

The output controller 31Dz outputs, as the analysis result, the histogram Hz generated by the statistical information generation unit 31Hz to the display device 35. The display form is the same as that in the first embodiment. That is, as illustrated in FIG. 8, the display is performed in the graph display region Ds3a set in the analysis result display region Ds3 of the sub-display region Ds.

### Processing Operation of Analysis

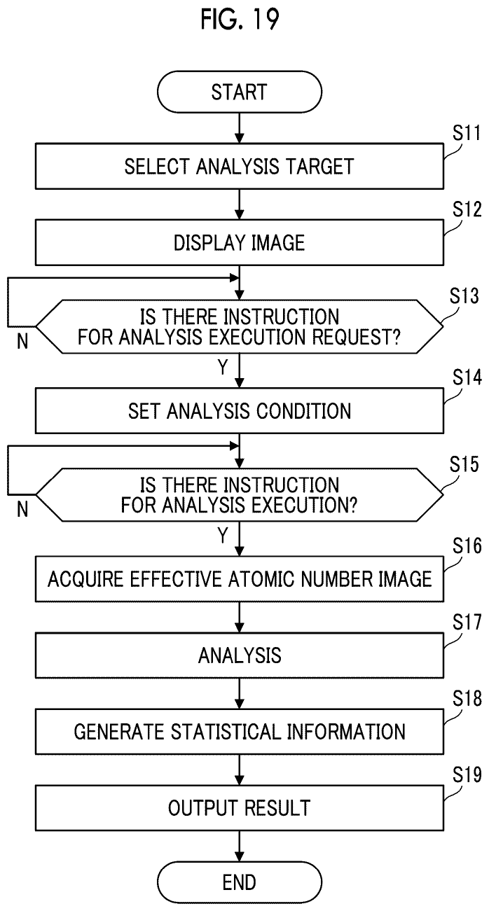

FIG. 19 is a flowchart illustrating an operation procedure of processing of obtaining the distribution of the effective atomic number.

First, an analysis target is selected (step S11). The analysis is performed on a series of effective atomic number images obtained in one examination. The user designates the examination as the analysis target, and selects an image group (series) that is the analysis target. The selection of the examination as the analysis target is performed on a predetermined selection screen.

In a case where the analysis target is selected, the image is displayed (step S12). Specifically, the effective atomic number image obtained by the examination designated as the analysis target is output to the display device 35 (refer to FIG. 5).

Next, it is determined whether or not there is an execution request of the analysis (step S13). In a case where the execution request of the analysis is received, the analysis condition is set (step S14). The user sets the analysis target region by adjusting the position or the like of the region setting frame displayed in a superimposed manner on the effective atomic number image on the screen. In addition, the user sets the range of the effective atomic number image as the analysis target, as necessary. For example, in a case of performing the analysis by narrowing down the range, the range of the effective atomic number image as the analysis target is set. After the setting is completed, the user gives an instruction to execute the analysis. The processor 31 determines whether or not there is an instruction to execute the analysis from the user (step S15).

In a case where the instruction to execute the analysis is given, the effective atomic number image as the analysis target is acquired (step S16). More specifically, a series of effective atomic number images as the analysis target is read out from the auxiliary memory 33. Then, the analysis processing is performed on the acquired series of effective atomic number images (step S17). Specifically, processing of counting the number of pixels for each effective atomic number is performed by targeting the analysis target region for each image.

In a case where the analysis of all the images is completed, the statistical information is generated on the basis of the analysis result (step S18). Specifically, a graph illustrating the distribution of the effective atomic number (frequency distribution of the presence amount for each effective atomic number) for each slice is generated. In the present embodiment, the bivariate histogram Hz illustrating the distribution of the effective atomic number for each slice is generated (refer to FIG. 18).

The generated histogram Hz is output, as the analysis result, to the display device 35 (step S19). In the present embodiment, the histogram Hz is displayed on the same screen as the screen on which the effective atomic number image as the analysis target is displayed (refer to FIG. 8).

As described above, the histogram Hz is composed of a three-dimensional graph in which the first horizontal axis H1 is the slice number, the second horizontal axis H2 is the effective atomic number, and the vertical axis V is the count number (presence amount) of the pixel (refer to FIG. 18). By checking the histogram Hz, it is possible to easily ascertain the distribution status of the effective atomic number for each slice. As a result, for example, it is possible to easily check which slice has the effective atomic number specific to the symptom. In addition, this improves the accuracy of image selection in the analysis.

### Modification Example

### Display of Analysis Result

As in the first embodiment, the histogram Hz of the analysis result may be displayed on the same screen as the screen on which the image for setting the analysis target region is displayed (refer to FIG. 8), or may be displayed on a different screen (different window) (refer to FIG. 10).

In addition, the histogram Hz may be generated with a numerical value of the largest effective atomic number among the effective atomic numbers extracted from the analysis target region as the display target.

In addition, the histogram Hz may be generated by targeting only the effective atomic numbers extracted from the analysis target region. That is, the histogram Hz in which only the effective atomic numbers extracted from the analysis target region are displayed on the second horizontal axis H2 may be generated.

### Designation of Display Range

### (1) Designation of Range of Effective Atomic Number to be Displayed

As in the first embodiment, in the histogram Hz to be displayed as the statistical information, the user may arbitrarily designate the range of the effective atomic number to be displayed (refer to FIGS. 11 and 12).

### (2) Setting of Threshold Value

The threshold value may be set, and the histogram Hz may be created by displaying, in an emphasized manner, the count number equal to or greater than the threshold value.

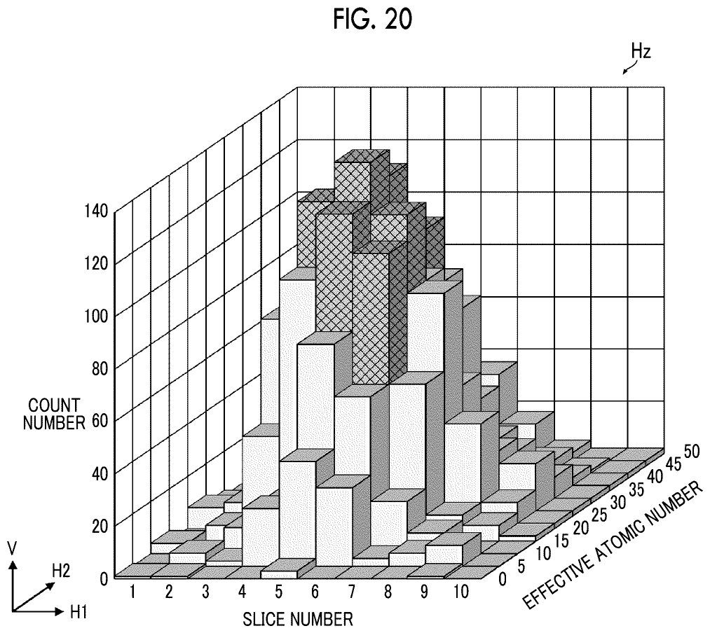

FIG. 20 is a diagram illustrating an example of the histogram displayed in an emphasized manner.

FIG. 20 illustrates an example of a case where bars of the effective atomic numbers of the count number exceeding the threshold value are displayed in an emphasized manner by changing the color of the bar.

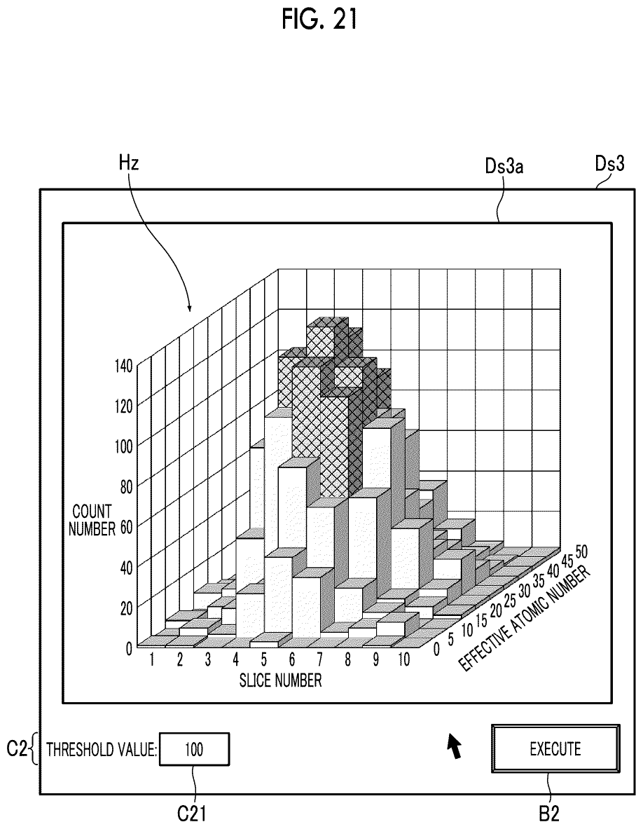

FIG. 21 is a diagram illustrating an example of a setting method of the threshold value.

As illustrated in FIG. 21, a field (threshold value setting field) C2 for setting the threshold value is provided in the analysis result display region Ds3. The threshold value setting field C2 includes a box C21 for inputting the threshold value.

In a case where the threshold value is set and the histogram Hz is displayed, the user inputs the threshold value to be set in the threshold value setting field C2, and clicks the execution button B2.

In this manner, by displaying, in an emphasized manner, the effective atomic number of the count number equal to or greater than the threshold value, it is possible to easily check the distribution of the effective atomic number.

### Provision of Analysis Function on Setting Screen of Post-Reconstruction

As in the case of the distribution analysis of the density value, it is preferable that the distribution analysis of the effective atomic number can also be performed on the post-reconstruction setting screen (refer to FIGS. 13 and 14).

### Provision of Analysis Function on Imaging Plan Screen

As in the case of the distribution analysis of the density value, it is preferable that the distribution analysis of the effective atomic number can also be performed on the imaging plan screen (refer to FIGS. 15 and 16).

### OTHER EMBODIMENTS

Distribution Analysis of Density Value and Distribution Analysis of Effective Atomic Number

In the same PCCT device 1, both the distribution analysis of the density value and the distribution analysis of the effective atomic number may be performed. Accordingly, it is possible for the user to select the analysis target according to the purpose, the use, and the like, and it is possible to further improve the accuracy of the image selection.

### Statistical Information

In the above-described embodiments, the configuration is adopted to generate the bivariate histogram using a so-called bar graph as the statistical information, but the form of the graph representing the statistical results is not limited to this.

For example, the statistical information may be generated using a heat map. The heat map is a visualization graph in which individual values of two-dimensional data (matrix) are represented as colors or shades. For example, for the distribution of the density values, a two-dimensional graph is generated by assigning colors or shades to the areas. Similarly, for the distribution of the effective atomic numbers, a two-dimensional graph is generated by assigning colors or shades to the count numbers.

### Spectral CT Device

In the above-described embodiments, a case where the present invention is applied to the PCCT device has been described as an example, but the application of the present invention is not limited to this. The present invention can be applied to any X-ray CT device capable of reconstructing the material discrimination image and/or the effective atomic number image from at least the detection data obtained by imaging. The spectral CT includes the detection of transmitted X-rays at two or more energy levels, and therefore, the spectral CT generally, by definition, includes dual-energy CT.

### Others

In the above-described embodiments, the console has the functions of the image processing and the analysis processing, but the functions of the image processing and the analysis processing may be provided by a device different from the console. In addition, a device that provides the function of the image processing and a device that provides the function of the analysis processing may be configured separately.

The processing unit that provides the function of the data processing apparatus can be configured by various processors. The various processors include, in addition to a CPU and a graphic processing unit (GPU) as a general-purpose processor, a programmable logic device (PLD) which is a processor of which the circuit configuration can be changed after manufacturing, such as a field programmable gate array (FPGA), and a dedicated circuitry which is a processor having a circuit configuration specifically designed to execute specific processing, such as an application specific integrated circuit (ASIC). One processing unit may be configured of one of various processors or may be configured of two or more processors of the same type or different types. For example, one processing unit may be configured by a combination of a plurality of FPGAs or a combination of a CPU and an FPGA. In addition, a plurality of processing units may be configured of one processor. As an example of configuring a plurality of processing units with one processor, first, there is a form in which, as typified by computers used for a client, a server, or the like, one processor is configured by combining one or more CPUs and software, and the processor functions as a plurality of processing units. Second, as typified by a system on chip (SoC) or the like, a processor that realizes the functions of the entire system including the plurality of processing units by using one integrated circuit (IC) chip is used. As described above, the various processing units are configured using one or more of the various processors as a hardware structure.

### EXPLANATION OF REFERENCES

1: PCCT device
10: scanner gantry
10A: opening portion
11: X-ray tube device
12: X-ray detection device
13: data acquisition system
14: rotation frame
20: examination table
21: top plate
30: console
31: processor
31A: data acquisition unit
31B: image processing unit
31C: recording controller
31D: output controller
31Dz: output controller
31E: image acquisition unit
31Ez: image acquisition unit
31F: analysis condition reception unit
31Fz: analysis condition reception unit
31G: image analysis unit
31Gz: image analysis unit
31H: statistical information generation unit
31Hz: statistical information generation unit
32: main memory
33: auxiliary memory
34: input device
35: display device
36: input and output interface
100: post-reconstruction setting screen
110: subject information display region
120: series information display region
130: post-reconstruction condition setting region
140: image display region
150: analysis condition setting region
160: post-reconstruction execution button
170: screen of analysis result
200: imaging plan screen
210: scanogram display region
220: subject information display region
230: imaging condition setting region
240: image display region
250: analysis condition setting region
260: OK button
270: close button
280: screen of analysis result
B1: density value distribution analysis button
B2: execution button
Bb: image backward button
Bf: image forward button
C1: display range setting field
C11: box for inputting start point of display range
C12: box for inputting end point of display range
C2: threshold value setting field
C21: box for inputting threshold value
Dm: main display region
Ds: sub-display region
Ds1: analysis menu display region
Ds3: analysis result display region
Ds3a: graph display region
F: region setting frame
H: histogram
Hz: histogram
I_n: material discrimination image
Im: reference image
MS: screen
P: subject
H1: first horizontal axis
H2: second horizontal axis
V: vertical axis
S1 to S9: operation procedure of processing of obtaining distribution of density values of material
S11 to S19: operation procedure of processing of obtaining distribution of effective atomic numbers

What is claimed is:

1. A data processing apparatus that processes data of a plurality of slices acquired by a spectral CT device, the data processing apparatus comprising:
a processor,
wherein the processor
outputs a first image generated on the basis of the data to a display destination,
receives setting of a region that is an analysis target, on the first image output to the display destination,
analyzes a second image of the plurality of slices generated on the basis of the data to generate statistical information indicating a distribution of a material in the region for each slice, and
outputs the statistical information to the display destination.

2. The data processing apparatus according to claim 1,
wherein the second image is a material discrimination image, and
the processor analyzes the material discrimination image of the plurality of slices generated on the basis of the data to generate statistical information indicating a distribution of a density value of the material in the region for each slice.

3. The data processing apparatus according to claim 2,
wherein the processor generates a histogram indicating an area of each density value in the region for each slice, as the statistical information.

4. The data processing apparatus according to claim 3,
wherein the processor
receives setting of a range of the density value to be displayed in the histogram, and
generates the histogram within the set range of the density value.

5. The data processing apparatus according to claim 2,
wherein the first image is a material discrimination image, and
the processor
outputs the material discrimination image generated on the basis of the data to the display destination, and
receives setting of the region on the material discrimination image output to the display destination.

6. The data processing apparatus according to claim 1,
wherein the second image is an effective atomic number image, and
the processor analyzes the effective atomic number image of the plurality of slices generated on the basis of the data to generate statistical information indicating a distribution of an effective atomic number in the region for each slice.

7. The data processing apparatus according to claim 6,
wherein the processor generates a histogram indicating a count number of each effective atomic number in the region for each slice, as the statistical information.

8. The data processing apparatus according to claim 7,
wherein the processor
receives setting of a threshold value, and
generates the histogram in which a portion corresponding to the count number equal to or greater than the threshold value is displayed in an emphasized manner.

9. The data processing apparatus according to claim 6,
wherein the first image is an effective atomic number image, and
the processor
outputs the effective atomic number image generated on the basis of the data to the display destination, and
receives setting of the region on the effective atomic number image output to the display destination.

10. The data processing apparatus according to claim 1,
wherein the processor
receives setting of a range of the second image that is the analysis target, and
analyzes the second image within the set range to generate the statistical information.

11. The data processing apparatus according to claim 1,
wherein the processor
outputs a screen for setting conditions for reconstruction in a case where the reconstruction is performed by changing the conditions, to the display destination, and
displays the first image on the screen.

12. The data processing apparatus according to claim 1,
wherein the processor
outputs a screen for setting conditions for imaging and reconstruction to the display destination, and
displays the first image on the screen.

13. The data processing apparatus according to claim 1,
wherein the spectral CT device is a photon counting CT device.

14. A data processing method of processing data of a plurality of slices acquired by a spectral CT device, the data processing method comprising:
a step of outputting a first image generated on the basis of the data to a display destination;
a step of receiving setting of a region that is an analysis target, on the first image output to the display destination;
a step of analyzing a second image of the plurality of slices generated on the basis of the data to generate statistical information indicating a distribution of a material in the region for each slice; and
a step of outputting the statistical information to the display destination.

15. A non-transitory, computer-readable tangible recording medium, which records thereon, a data processing program for processing data of a plurality of slices acquired by a spectral CT device, the data processing program causing a computer to implement:
a function of outputting a first image generated on the basis of the data to a display destination;
a function of receiving setting of a region that is an analysis target, on the first image output to the display destination;
a function of analyzing a second image of the plurality of slices generated on the basis of the data to generate statistical information indicating a distribution of a material in the region for each slice; and
a function of outputting the statistical information to the display destination.
